(19) United States
(12) Patent Application Publication
(10) Pub. No.: US 2021/0299400 A1
(43) Pub. Date: Sep. 30, 2021

(54) ASSEMBLY FOR DELIVERING AN AGENT

(71) Applicants: BRUSSELS MEDICAL DEVICE CENTER ASBL, Brussels (BE); UNIVERSITE LIBRE DE BRUXELLES, Brussels (BE)

(72) Inventors: Nicolas CAUCHE, Anderlecht (BE); Cecilia DELATTRE, Anderlecht (BE); Alan LINTERMANS, Anderlecht (BE); Jean MONFILS, Bruxelles (BE); Jacques DEVIERE, Bornival (BE)

(21) Appl. No.: 17/344,764

(22) Filed: Jun. 10, 2021

Related U.S. Application Data
(63) Continuation-in-part of application No. PCT/EP2019/084483, filed on Dec. 10, 2019.

(30) Foreign Application Priority Data
Dec. 10, 2018 (EP) 18211438.9

(51) Int. Cl. A61M 25/00

(52) U.S. Cl. CPC A61M 25/007; A61M 25/0075; A61M 2025/0073; A61M 2025/0004; A61M 25/003

(57) ABSTRACT

An assembly for delivering a therapeutic agent includes a catheter and a supply system. The catheter includes a catheter body having a first lumen extending from a proximal end to a distal end and defining a longitudinal axis, and a first outlet adjacent to the distal end and in fluid communication with the first lumen. The supply system includes an inlet port for a carrier fluid, an inlet port for the agent, and an output port in fluid communication with the two inlet ports and the first lumen at the proximal end. The supply system further includes a fluid path from one inlet port to the output port having a constriction in vicinity of the other inlet port configured to aspirate the agent by Venturi effect. The catheter includes a valve arranged adjacent the one outlet and in fluid communication with the first lumen.

### CROSS-REFERENCES

The following applications and materials are incorporated herein, in their entireties, for all purposes: International Publication No. WO2020120498A1, filed Dec. 10, 2019 as PCT/EP2019/084483. However, such material is only incorporated to the extent that no conflict exists between the incorporated material and the statements and drawings set forth herein. In the event of any such conflict, including any conflict in terminology, the present disclosure is controlling.

### FIELD

The present disclosure is related to assemblies for delivering an agent, such as a drug or other therapeutic agent, into an internal cavity of a human or animal body. The agent is supplied in powder form using a carrier fluid. The present disclosure is particularly related to assemblies for delivering an agent in the gastro-intestinal (GI) tract.

### INTRODUCTION

Assemblies for delivering therapeutic agents in powder form are known from US 2009/0281486, 12 Nov. 2009, and US 2015/0094649, 2 Apr. 2015. These assemblies comprise a container for holding the therapeutic agent and a source of pressurized fluid. Both are fluidly coupled to a catheter through a connecting member having a first inlet port coupled to the container, a second inlet port coupled to the source of pressurized fluid, and an outlet port coupled to the catheter. The connecting member exploits Bernoulli's principle of fluid dynamics to provide a localized low-pressure system in the vicinity of the first inlet port. The pressurized fluid passing through the connecting member will form a strong suction force when it passes the first inlet port. As a result, the therapeutic agent is suctioned from the first inlet port towards the catheter.

One problem that has been observed when using assemblies of the above kind, is that the catheter has tendency to clog in the distal region.

Gastrointestinal bleeding, in particular in the esophagus and the stomach, can have dramatic consequences if not timely treated. Medical guidelines advocate a treatment within 12 hours after patient presentation. However, data has shown that a significant portion of the patients have a delay greater than 24 hours before undergoing endoscopy. The mortality rate of acute variceal bleeding in the upper GI tract is around 20% at six weeks. (Source: Early application of hemostatic powder added to standard management for oesophagogastric variceal bleeding: a randomised trial, Mostafa Ibrahim et al., Gut 2018; 0:1-10; doi:10.1136/gutjnl-2017-314653)

A first aid measure to stop gastrointestinal bleeding is to supply a hemostatic agent to the bleeding site, mostly in powder form. It has been found that early (e.g., within two hours) application of hemostatic powder improves clinical and endoscopic hemostasis. However, when the catheter gets clogged, this causes a serious risk for the patient, as an insufficient amount of hemostatic powder will be delivered.

Yet another difficulty is that such catheters require vision of the internal lumen to deliver the powder at the adequate site. To this end, the presence of an endoscopist is required. However, a patient may need to wait a long time for such trained personnel to be available, which may lead to increasing mortality probabilities. Yet another difficulty is that some sites in the GI tract, in particular in the stomach are difficult to reach by the spray holes of the catheter. These sites are located at the rear of the catheter and would require the catheter to be bent backwards. However, without vision as through an endoscope, this would result in a difficult and time-consuming task.

### SUMMARY

It is therefore an aim of the present disclosure to provide an assembly allowing to deliver an agent, in particular in powder form, into a human or animal body that overcomes one or more of the above problems. It is an aim to provide such assemblies that are more reliable in delivering therapeutic agents, in particular that reduce a risk of clogging of the catheter by the agent that is delivered. It is an aim to enable to easily supply a desired agent even in endoluminal areas which are difficult to reach and/or without requiring vision anymore. It is therefore an aim of the present disclosure to provide assemblies for delivering an agent inside a body lumen which would possibly not require a trained endoscopist. It is an aim to provide such assemblies allowing a faster and more effective delivery of an agent in order to stop bleeding earlier and potentially give higher survival rates to the patients, in particular in case of gastrointestinal bleeding.

Assemblies according to the present disclosure comprise a catheter, which comprises a catheter body comprising a first lumen extending from a proximal end to a distal end. The first lumen defines a longitudinal axis, which may be identical to a longitudinal axis of the catheter body. The catheter further comprises a first outlet adjacent to the distal end and in fluid communication with the first lumen.

According to a first aspect, the assembly comprises a supply system, in particular for delivering a mixture of the agent and a carrier fluid, particularly a gas. The supply system comprises a first inlet port for a carrier fluid, a second inlet port for the agent and an output port in fluid communication with the first and second inlet ports and the first lumen at the proximal end. The supply system comprises a fluid channel or path arranged from the first inlet port to the output port, the fluid channel or fluid path comprising a constriction in vicinity of the second inlet port configured to aspirate the agent from the second inlet port through the Venturi effect. The constriction portion of the fluid channel or fluid path is referred to hereinafter as a Venturi channel.

According to a second aspect, which can be provided in conjunction with, or independent of the first aspect above, the catheter comprises a valve configured to close the first lumen from the external environment. The valve may close the first lumen at the distal side, such as at the first outlet, or further upstream. The valve is advantageously a pressure-sensitive valve, which opens upon reaching a threshold (differential) pressure level in the first lumen. To this end, the valve may comprise a valve seat and a pressure-sensitive valve member, such as a resilient and/or flexible membrane, which advantageously surrounds the valve seat. The valve may be configured to close again once the (differential) pressure in the first lumen falls below the threshold.

With such a valve system, it becomes possible to close the first lumen to avoid exposure of the agent within the first lumen to bodily fluids and moisture during introduction and manipulation of the catheter in the body. As a result, a risk of clogging of the lumen due to reaction of the (powdery) agent with the external fluids is greatly reduced or avoided.

It has been surprisingly found that the combination of the supply system according to the first aspect above and the valve according to the second aspect above provides synergistic effects, in that the supply system prevents or at least reduces clogging of the lumen at the distal side. Furthermore, in case a clogging would occur, the combination of valve and supply system as described above reduces the size of the clog and aids in evacuating the clog from the lumen. The supply system will only aspirate agent from its reservoir into the catheter when a flow of carrier fluid is created, which occurs when the valve opens. Therefore, a heaping up of agent in the lumen is prevented, which greatly reduces clogging. In case a clog would occur in the lumen of the catheter, the flow of carrier fluid will stop, preventing agent to heap up at the clog. The clog therefore does not increase in size. Simultaneously, the pressure in the lumen will increase, which aids in evacuating the clog from the lumen. The supply system therefore allows for using a pressure-sensitive valve system in assemblies as described herein in a reliable way.

Advantageously, the pressure sensitive valve member is a resilient membrane circumferentially attached to the catheter body. Advantageously, the resilient membrane comprises an outer edge defining a circumference of the resilient membrane. A portion of the resilient membrane, e.g., adjacent to the outer edge, can be shaped as a possibly substantially cylindrical sleeve which is made to seal against the catheter body, e.g., at a periphery or circumference thereof. The sealing portion of the sleeve hence is attached to the catheter body. Advantageously, the resilient membrane comprises a through-opening and an inner edge of the membrane defines a perimeter of the through-opening. The inner edge seals against the valve seat in a closed state of the valve and is deflected from the valve seat in an open state of the valve. Advantageously, in a closed state of the valve, the inner edge is axially spaced apart from the attachment portion of the resilient membrane to the catheter body. When the valve member is attached along a perimeter or circumference of the catheter body, and a central through-opening is provided, an improved deformation of the valve member due to the differential pressure is obtained. The valve member will deform through one or a combination of axial elongation and radial bulging.

Advantageously, in the supply system, the second inlet port and the output port have parallel axes, which may or may not be offset from each other. It has been observed that with such a disposition of the ports, an optimal Venturi effect of aspiration of agent powder can be obtained, which furthermore prevents clogging of the supply system itself, and of the lumen further downstream. The first inlet port can have an axis which is parallel to the axis of either one or both the second inlet port and the output port. Advantageously, the Venturi channel and the second inlet port are concentric. It is however alternatively possible to arrange the Venturi channel and the second inlet port eccentric with respect to one another. With any of the above dispositions in the supply system, it has been observed that a carrier fluid/agent mixture having a higher agent to fluid ratio can be generated. Mixtures with relatively higher amount of agent in a comparable fluid volume are preferred since this will lead to reduced insufflation dangers when treating a patient. It will be convenient to note that supply systems according to aspects of the present disclosure can be easily adjusted for use with powders of differing particle size.

According to a third aspect, which can be provided in combination with the first and second aspects above, or in isolation, the catheter comprises fluid deflecting means configured to deflect a flow of a (medicinal or therapeutic) agent discharged from the first outlet in a backwards and possibly radial direction relative to the flow of the agent in the first lumen. The (mean) velocity of the flow deflected by the fluid deflecting means hence has a component along the longitudinal axis oriented backwards relative to the flow through the first lumen, the latter being directed towards the proximal end. In addition, the (mean) flow velocity can have a normal component relative to the longitudinal axis (i.e., a radial component). Advantageously, the fluid deflecting means comprises a fluid deflecting surface, which is advantageously arranged opposite (i.e., facing) an outlet of the first lumen, which can be the first outlet, or an additional outlet. Advantageously, the fluid deflecting surface is inclined with respect to the longitudinal axis at an angle from 95° to 180°, advantageously from 105° to 175° measured from a distal side of the longitudinal axis. The fluid deflecting surface can be mounted at a fixed orientation, or can be movable between different orientations relative to the longitudinal axis, e.g., assuming a deployed configuration with an orientation as indicated above and a non-deployed configuration, e.g., for reducing bulkiness.

Such a disposition of the fluid deflecting surface allows for deflecting the agent delivered through the catheter in a backwards and possibly radial direction, so that areas at the rear of the catheter can be reached by the agent easily and without need of bending or excessively manipulating the catheter body. It will be convenient to note that bending a catheter, such as those in the prior art, would increase its likeliness to jam and clog.

Advantageously, the first fluid deflecting means is formed of, or comprises a fluid channel or chamber extending between an outlet of the first lumen and the first outlet, the latter being arranged at a proximal side compared to the outlet of the first lumen. The outlet of the first lumen hence forms an inlet port to the fluid channel. The outlet of the first lumen can be arranged in a side wall of the first lumen, or at a distal end of the first lumen. The fluid channel advantageously extends parallel to the first lumen (e.g., parallel to the longitudinal axis). Advantageously, the fluid channel can completely surround the first lumen, e.g., the first channel can be concentric with the first lumen. With such arrangement, a wall of the fluid channel opposite the first outlet acts as fluid deflecting surface. By way of example, the first fluid deflecting means can be configured to impart a 180° change of flow direction to the flow discharged from the first lumen.

Advantageously, at the first outlet, possibly arranged downstream of the outlet of the first lumen, a further fluid deflecting step can be provided, hence, downstream of the (first) fluid deflecting surface. The second fluid deflecting step can be configured to impart a change of flow direction in an opposite sense compared to the directional change imparted by the first fluid deflecting means, e.g., by an angle from 5° to 85°, advantageously an angle from 15° to 65° in an opposite sense compared to the flow direction imparted by the first fluid deflecting means. As a result, a radial component in addition to the backwards component is imparted to the fluid. A total change of flow direction at the first outlet compared to the direction of flow in the first lumen can be from 95° to 175°.

To provide the second fluid deflecting step, the fluid deflection means can comprise a protrusion extending radially of the catheter body. The protrusion is advantageously arranged opposite the first outlet. The protrusion comprises a fluid deflecting surface opposite the first outlet that acts as a fluid impingement surface and configured to impart the above indicated change in flow direction.

Such a second fluid deflecting means hence allows for obtaining a two-step fluid deflection, which makes the deflection at the fluid deflecting surface easier and more efficient. Moreover, it becomes possible to protect the flow of agent in the second fluid deflecting means from the external environment more effectively, leading to improved operational performance and less risk of clogging due to exposure to external factors.

Methods of treating a medical condition of a human or animal body are described. These methods operate by supplying an agent in powder form utilizing assemblies as described herein. Possible medical conditions are the treatment of gastrointestinal bleeding resulting from endoscopic treatments such as treatment of diverticulum, polyps or fistula or resulting from spontaneous bleedings due to ulcers, tears or varices. Assemblies according to aspects of the present disclosure can be utilized in other body parts than the GI tract, for example treatment in the urinary tract, of the sex organs, and of the respiratory tract, in particular the trachea and the lungs (bronchi, alveolar duct, alveolar sacs). It will be convenient to note that the catheter can also be used for veterinary applications.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

Aspects of the present disclosure will now be described in more detail with reference to the appended drawings, wherein same reference numerals illustrate same features and wherein:

FIG. 1 represents an overview of parts of an assembly according to aspects of the present disclosure;

FIG. 2 represents a side view of a delivery system for endoluminal delivery of an agent according to a first embodiment;

FIG. 3 represents a cross sectional view of the delivery system of FIG. 2 along section line A-A;

FIG. 4 represents a cross sectional view of an alternative delivery system according to the present disclosure;

FIG. 5 represents a cross sectional view of the distal end of a catheter comprising two delivery systems connected to a same delivery lumen, for spraying an agent in backward and forward direction;

FIG. 6 represents a side view of a delivery system for endoluminal delivery of an agent according to a second embodiment;

FIG. 7 represents a cross sectional view of the delivery system of FIG. 6 along section line B-B;

FIG. 8 represents a perspective view of a delivery system for endoluminal delivery of a medicinal agent according to a third embodiment;

FIG. 9 represents a cross sectional view of the delivery system of FIG. 8 along section line C-C;

FIG. 10 represents a perspective view of a delivery system for endoluminal delivery of a medicinal agent according to a fourth embodiment;

FIG. 11 represents a cross sectional view of the delivery system of FIG. 10 along section line D-D;

FIG. 12 represents a cross sectional view of a supply system according to a first embodiment, for use with the delivery systems as described herein;

FIG. 13 represents a view of the stomach with a catheter according to aspects of the present disclosure inserted therein;

FIG. 14 represents a schematic view of another assembly according to aspects of the present disclosure;

FIG. 15 represents a perspective and partial cut-out view of a delivery system of an agent according to a fifth embodiment suitable for use in the assembly of FIG. 14;

FIG. 16 represents an axial sectional view of the delivery system of FIG. 15 in a closed state of the valve;

FIG. 17 represents an axial sectional view of the delivery system of FIG. 15 in an open state of the valve;

FIG. 18 represents a perspective and partial cut-out view of the delivery system according to another exemplary embodiment;

FIG. 19 represents a perspective and partial cut-out view of the delivery system according to yet another exemplary embodiment;

FIG. 20 represents a cross sectional view of a supply system according to a second embodiment, for use with the delivery systems as described herein;

FIG. 21 represents a cross sectional view of a supply system according to a third embodiment, for use with the delivery systems as described herein;

FIG. 22A represents a perspective view of yet another embodiment of a delivery system according to aspects of the present disclosure, including an inflatable balloon represented in an inflated state; and

FIG. 22B represents a plan view of the delivery system of FIG. 22A.

### DETAILED DESCRIPTION

Various aspects and examples of assemblies for delivering an agent, such as a drug or other therapeutic agent, into an internal cavity of a human or animal body, as well as related methods, are described below and illustrated in the associated drawings. Unless otherwise specified, an assembly in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

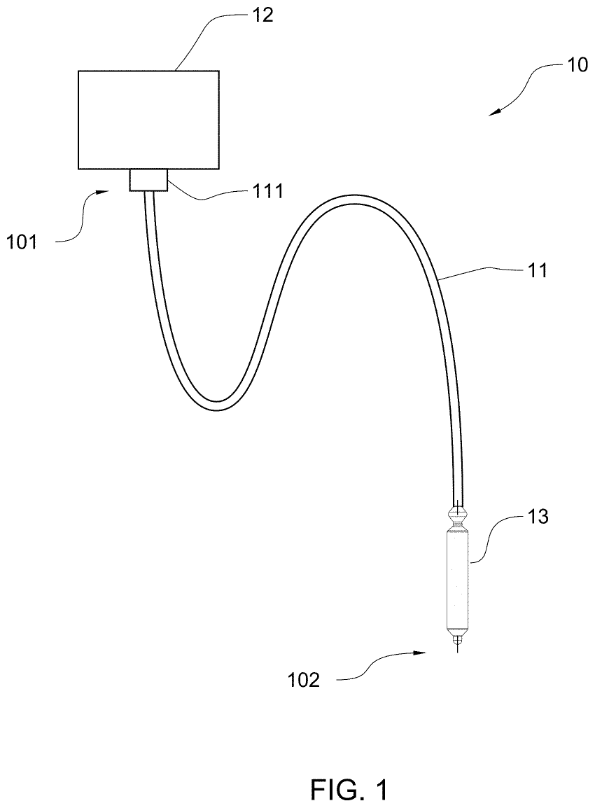

Referring to FIG. 1, an assembly 10 according to aspects of the present disclosure comprises a catheter body 11 that extends from a proximal end 101 to a distal end 102. The catheter body 11 is advantageously configured to be inserted in a lumen or cavity of a human or animal body through natural orifices, such as through the nasal cavity, or can be inserted through the instrument channel of an endoscope. The catheter body 11 can comprise attachment means, such as a connector 111, at the proximal end 101 for attachment to a supply system 12 for supplying a medicinal or other, e.g., therapeutic, agent. At the distal end 102, the catheter body 11 is provided with a delivery system 13 for delivering the agent to the body. The catheter body 11 comprises a delivery lumen extending from the proximal end 101 and in fluid connection with the supply system 12, to the delivery system 13.

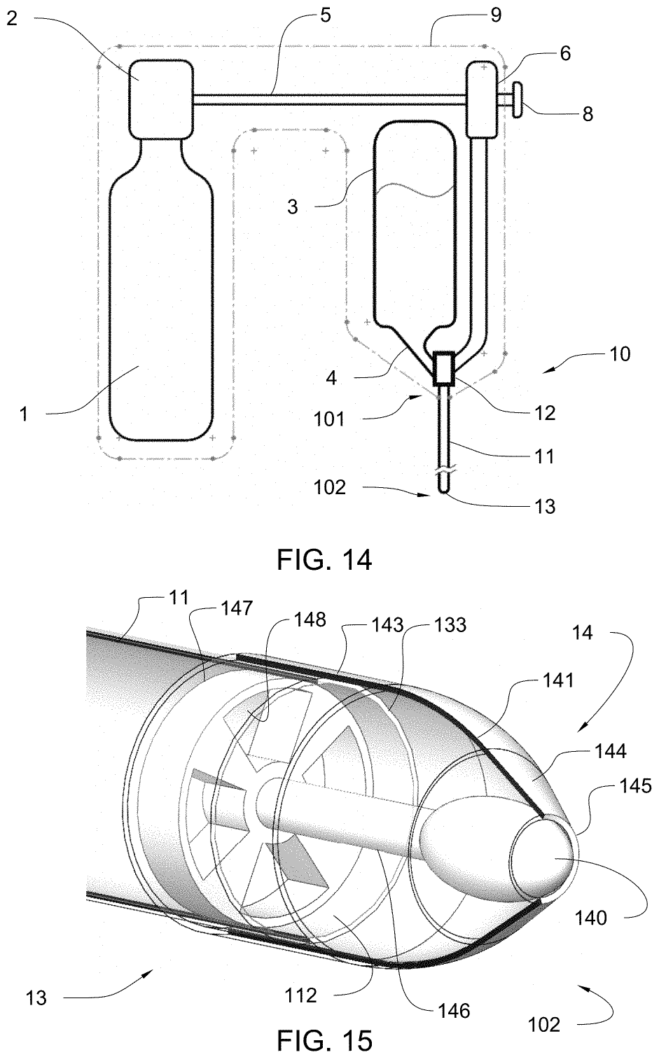

Referring to FIG. 14, the assembly 10 can be coupled to a source 1 of a carrier fluid, in particular a carrier gas, such as CO2 gas, which is advantageously pressurized. The source 1 is fluidly coupled to a first inlet port of the supply system 12 through suitable duct system 5 and possibly a pressure regulating valve 2. A reservoir 3 comprising the agent to be delivered is fluidly coupled to a second inlet port of the supply system 12 through a suitable duct system 4. The supply system 12 is configured to withdraw agent from reservoir 3 and have it transported to the catheter body 11 by means of the carrier fluid, as will be described further below.

The assembly can be operated through a switch valve 6 arranged in the fluid path (duct system 5) of the carrier fluid, advantageously upstream of the first inlet port of the supply system 12. Switch valve 6 is advantageously a normal closed valve closing the fluid path of the carrier fluid which interrupts the flow of agent from the reservoir 3 to the catheter body 11. Switch valve 6 can comprise a push button 8 allowing to open the fluid path of the carrier fluid, which starts the flow of agent towards the catheter body 11. The source 1, reservoir 3 and supply system 12 can be formed as a handheld part 9, whereas the catheter body 11 and delivery system 13 are configured for insertion into a patient.

Referring to FIGS. 15, 16 and 17, the delivery system 13 is arranged at a distal end 102 of the catheter body 11. Delivery lumen 112 extends through the catheter body 11 until the distal end 102, where an outlet 133 of delivery lumen 112 is provided. Delivery lumen 112 and catheter body 11 define a longitudinal axis 103 and is configured to transport the agent to a treatment site inside the body. A valve 14 is arranged at outlet 133 and is operable to close the outlet 133 and hence the delivery lumen 112 from ambient. The valve 14 advantageously prevents bodily fluids and moisture to enter the catheter and the delivery system 13 in particular during insertion of the catheter in the body and during manipulation of the catheter until it reaches the desired treatment site. This is particularly advantageous when the agent that is to be administered is in powder form and may react under influence of these external fluids, as there would be a risk of clogging the catheter if the bodily fluids or moisture would enter the catheter.

A resilient membrane 141 forms the valve member and is occluding the outlet 133 in a closed state of the valve 14. Resilient membrane 141 advantageously comprises a sleeve portion 143 which is attached to the catheter body 11, e.g., to a side wall of catheter body 11. A constricting portion 144 of the resilient membrane 141 extends from the sleeve portion 143 towards a valve seat 140. A through-opening 149 is formed through resilient membrane 141. The perimeter of the through-opening forms an inner edge 145 which rests in sealing contact on the valve seat 140 when the valve is in closed state. In the figures, the constricting portion 144 is represented with substantially conical or frusto-conical shape, although other shapes, such as substantially spherical are possible as well.

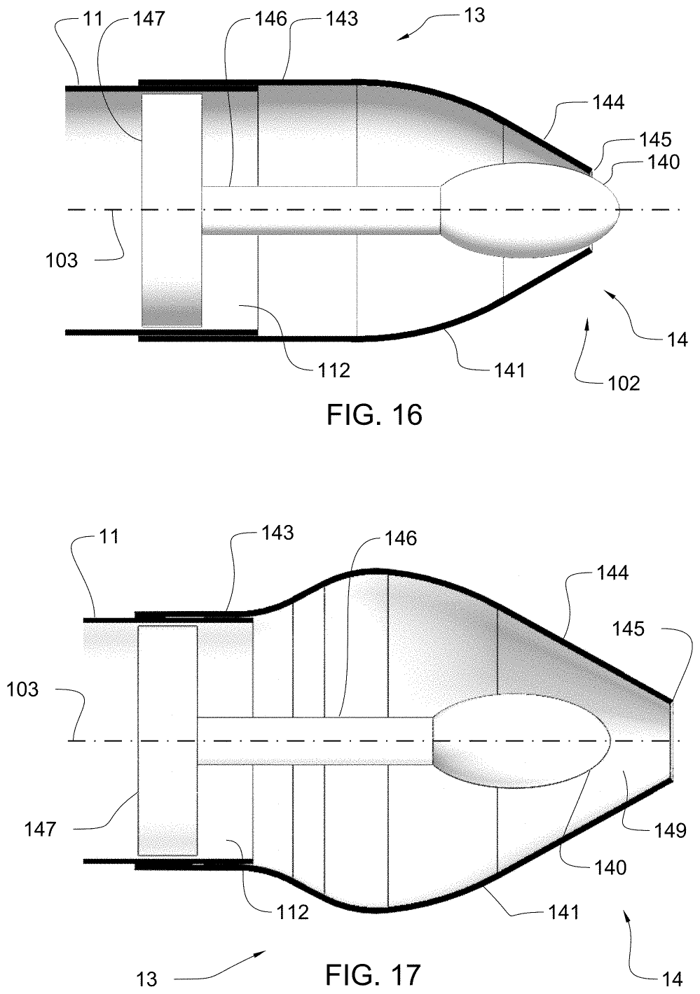

As shown in detail in FIG. 16, in a closed state of valve 14, the constricting portion 141 tapers from the sleeve portion 143 of the resilient membrane towards the valve seat 140, where a seal is formed against the valve seat 140. When pressure in the delivery lumen increases, the resilient membrane 141 advantageously experiences a force by the fluid pressure in the delivery lumen which can act in distal (axial) direction, towards the valve seat 140, in radial direction towards the outside or both axially and radially. As a result, as shown in FIG. 17, the resilient membrane may dilate in axial direction and/or bulge in radial direction. When a differential pressure in delivery lumen 112 as compared to the pressure at the outside of the delivery system 13, e.g., at the treatment site, exceeds a threshold, which may be predetermined, the inner edge 145 detaches from the valve seat causing the valve 14 to open. Valve 14 is hence advantageously a pressure sensitive valve.

For a pressure-sensitive valve, the threshold pressure level for opening the valve can advantageously be set to a working pressure level of the supply system 12, e.g., from 10 mbar to 1 bar or from about 1 bar to about 8 bar, such that the valve opens when the supply system is turned on.

The valve seat 140 can have any suitable shape, such as spherical or conical, advantageously with rounded (atraumatic) tip. It advantageously has a circular cross section in a plane perpendicular to the longitudinal axis 103. The valve seat 140 protrudes towards a distal side from the outlet 133 and can be fixedly attached to the catheter body 11 while enabling a fluid passage from the outlet 133 to ambient when the valve is open.

In one exemplary embodiment, as shown in FIGS. 15-17, the valve seat 140 is attached at a tip end of a rod 146, which in turn is attached to an attachment member 147 attached to an internal wall of the delivery lumen 112. The attachment member 147 comprises through openings 148 to allow passage of the carrier fluid and the agent towards the outlet 133.

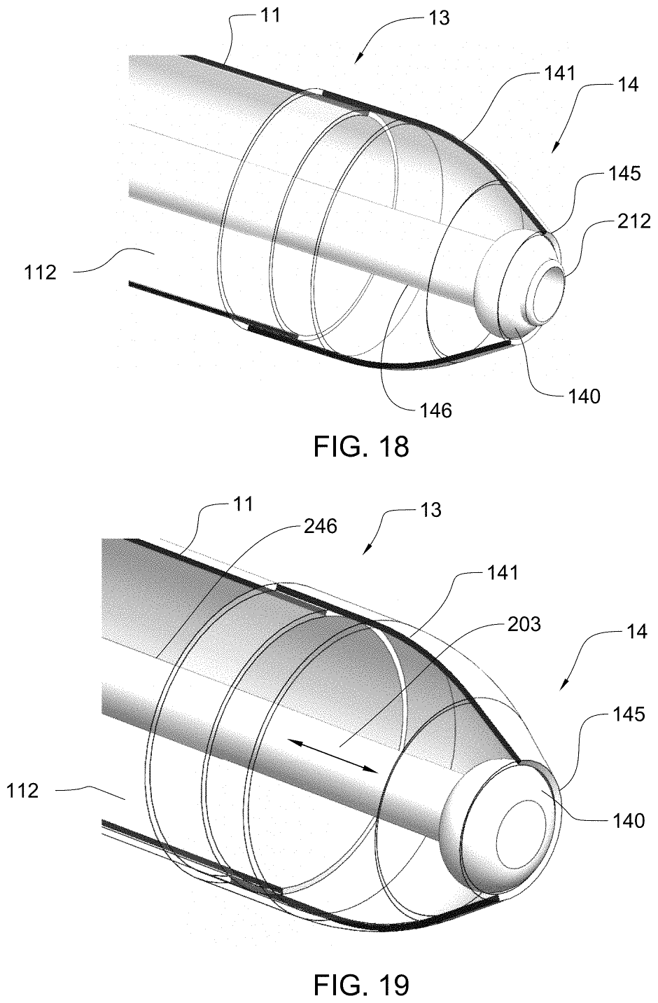

Referring to FIG. 18, the rod 146 can be hollow and extend distally past the valve seat 140 and proximally inside the delivery lumen 112 until the proximal end. The valve member 141 and the valve seat can be identical to the embodiments of FIGS. 15-17. The hollow rod 146 advantageously creates a lumen 212 for passing a guide wire or any other suitable instrument. Lumen 212 is advantageously fluidly isolated from the delivery lumen 112 and does not hinder operation of the valve 14. Lumen 212 can be used as a channel for suction, for insufflation, or for injection of any useful substance as desired.

Referring to FIG. 19, in a yet alternative embodiment, the valve member 14 is identical to the previous embodiments, while the valve seat 140 is attached to the distal end of a rod 246 which is moveable relative to the catheter body 11 in axial direction as indicated by the arrow 203. Rod 246 can e.g., be slideable relative to a bearing member (not shown) provided in the delivery lumen 112 and attached to the catheter body 11. Such bearing member can be substantially similar to the attachment member 147 in FIG. 15, with the difference that rod 246 is configured to slide axially in the bearing member. Rod 246 advantageously extends through delivery lumen 112 until the proximal end. In such case, rod 246 can be formed as a push rod, which may be securable in one or more axial positions relative to delivery lumen 112. This can be useful for enlarging or opening of the valve 14, e.g., for temporarily boosting the flow of agent delivered to the target site by pulling the rod proximally so that the valve seat separates from the valve member. On the other hand, the rod may be pushed in distal direction, e.g., to ensure a better closing of the valve.

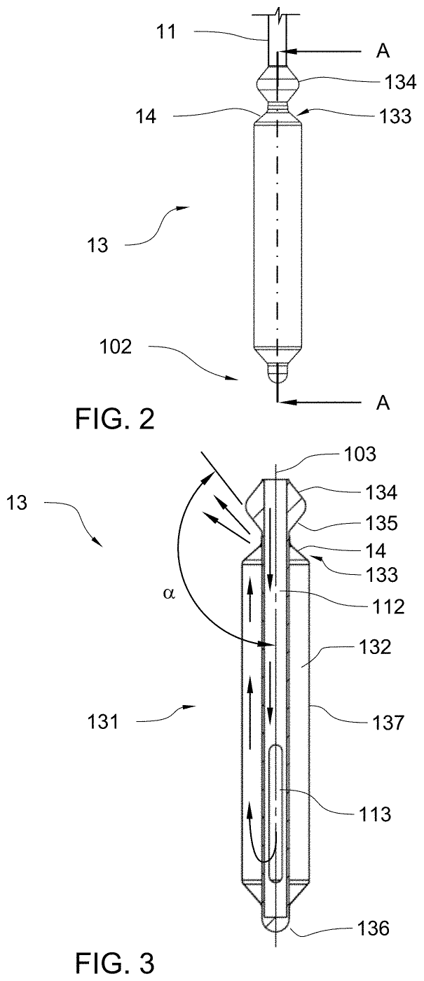

The delivery systems of the embodiments set forth hereinabove allow for delivering or spraying the agent in a forward (distal) direction, compared to the flow in the delivery lumen. The present disclosure however also contemplates delivery systems allowing backwards spraying of the agent. Referring to FIGS. 2 and 3, another embodiment of delivery system 13 has delivery lumen 112 extending through the catheter body 11 until the distal end 102. Lumen 112 defines a longitudinal axis 103 and is configured to transport the agent to a treatment site inside the body. Lumen 112 advantageously comprises a closed tip 136 at the distal end. The delivery system 13 comprises a fluid deflecting system 131 configured to deflect the forward flow of agent to a radial and backward flow. Fluid deflecting system 131 comprises a fluid channel or chamber 132 which is arranged at a circumference of lumen 112 and advantageously completely surrounds lumen 112 at the distal end. Lumen 112 comprises an outlet 113 for fluid communication with fluid chamber 132. Outlet 113 can be formed as one or more slots or holes provided in a wall of the lumen 112. Multiple of such slots or holes may be arranged at different angular positions around the wall of lumen 112. Alternatively, or in addition, it may be possible to open the tip 136 of lumen 112 towards fluid chamber 132.

The fluid chamber 132 extends from the tip 136 of lumen 112 to the outlet 133 of the delivery system 13 arranged at a proximal side compared to tip 136. The outlet 133 is advantageously provided at an end of the fluid chamber 132 opposite the outlet 113 of delivery lumen 112. Outlet 133 opens to the body lumen or cavity to deliver the agent.

The delivery system 13 as shown in FIGS. 2 and 3 advantageously has rotational symmetry about axis 103, e.g., symmetry with respect to a rotation over an angle of 180°, 120°, 90° or 45°. It may be convenient to make outlet 133, as well as fluid impingement surface 135 as a circular aperture surrounding lumen 112 in order to deliver a medicinal agent in 360° directions around axis 103.

The agent, in gaseous, liquid or powder form is advantageously transported through lumen 112 with a carrying fluid, which may be liquid or gaseous, such as CO2. An appropriate fluid/agent mixture is advantageously prepared in supply system 12. At the distal end, the fluid/agent mixture exits lumen 112 through outlet 113 and enters fluid chamber 132. An external wall 137 of fluid chamber 132 is advantageously arranged opposite outlet 113 to deflect fluid and agent discharged from the outlet 113 towards the outlet 133. The fluid/agent mixture then flows from delivery lumen outlet 113 to outlet 133 from where it is discharged. The flow direction of the fluid/agent mixture hence makes a 180° turn in proximity of the outlet 113, since the fluid flow in the fluid chamber 132 will be in opposite direction with respect to the fluid flow in lumen 112, as indicated by the arrows in FIG. 3. At the outlet 133, the fluid/agent mixture is discharged from fluid chamber 132 to advantageously reach treatment sites which are located behind the delivery system 13.

A fluid deflecting member 134 is advantageously arranged adjacent to outlet 133. Fluid deflecting member 134 comprises a fluid impingement surface 135 facing the outlet 133. Fluid that exits the fluid chamber 132 though outlet 133 impinges on surface 135 and is deflected, advantageously in a radial direction. Fluid impingement surface 135 therefore adds a further directional change to the fluid flowing through the fluid chamber 132, e.g., to increase the spray angle. Fluid impingement surface 135 is advantageously inclined relative to the longitudinal axis 103 at an angle α advantageously falling in a range from 95° to 175°, advantageously from 100° to 170°, advantageously from 105° to 165°, advantageously from 110° to 160°, advantageously from 115° to 155° measured as from a distal (forward) side of the longitudinal axis 103. Doing so, a broad spray angle is advantageously obtained allowing for effectively treating areas of the GI tract that are located at the rear of the delivery system 13 and without having to bend or deflect the catheter body. Endoscopic vision during delivery of the agent is not required since it is ensured that the target areas located at the rear of the delivery system are treated. Furthermore, catheter manipulation is eased, reducing treatment time.

A valve 14 is advantageously arranged at the outlet 133, or possibly at an upstream location in fluid chamber 132. Valve 14 is advantageously a pressure-sensitive valve arranged to open the outlet 133 when a predetermined pressure level is reached in fluid chamber 132. Alternatively, valve 14 can be a remotely actuated valve, e.g., connected to the proximal end, such as through an actuating cable, to open the valve 14 when desired. The operation of valve 14 is similar to the valve described in relation to FIG. 18 above, where the lumen 212 would be the delivery lumen 112 and delivery lumen 112 would be replaced by the fluid chamber 132.

As an alternative, a seal can be provided instead of valve 14 to close the outlet 133. The seal is advantageously pressure-sensitive, in the sense that it opens the outlet once a predetermined pressure level is attained in the fluid chamber 132. A seal is typically for single use, as it may not able to close the outlet again at the end of treatment and/or when pressure in the fluid chamber 132 is reduced, e.g., the seal is frangible at a predetermined pressure level.

In a yet alternative case, the valve 14, or a seal can be provided at the outlet 113 of delivery lumen 112, instead of the outlet 133.

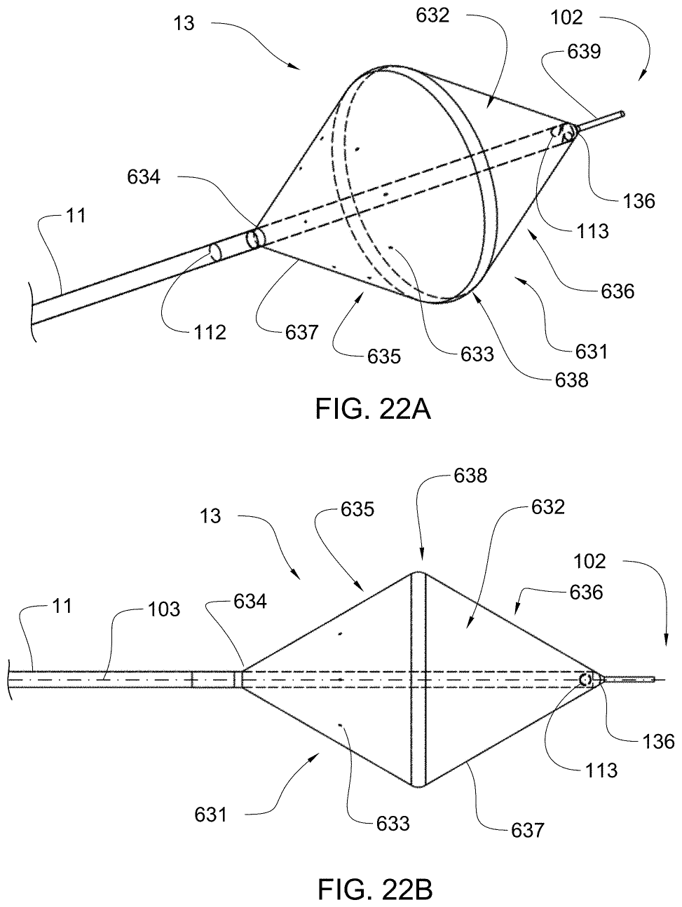

Referring to FIGS. 22A-22B, another embodiment of delivery system 13 has delivery lumen 112 extending through the catheter body 11 until a distal tip 136. The delivery system 13 comprises a fluid deflecting system 631 configured to deflect the forward flow of agent through delivery lumen 112 to a radial and backward flow of agent egressing from an outlet of the delivery system 13. Fluid deflecting system 631 comprises a fluid chamber 632 which is arranged at a circumference of delivery lumen 112 and advantageously completely surrounds delivery lumen 112 at the distal end.

Delivery lumen 112 can be closed at the distal tip 136, while one or a plurality of through holes 113 in a sidewall of catheter body 11 adjacent the distal tip 136 form a fluid communication path between delivery lumen 112 and fluid chamber 632. These through holes 113 form an egress port of delivery lumen 112. Multiple through holes 113 may be arranged at different angular positions about axis 103. By way of example, through holes 113 can have their centers located from 5 to 20 mm from tip 136 as measured along axis 103. Through holes 113 may have a diameter ranging from 1 to 5 mm, advantageously from 1.5 mm to 3 mm. The through holes can have any suitable shape, e.g., circular, oval or elliptical with longer axis parallel to axis 103. Possibly, delivery lumen 112 may continue distally of tip 136, e.g., towards a further delivery system located further downstream (not shown in FIGS. 22A-22B).

The fluid chamber 632 extends from the tip 136 to past a proximal side of the through holes 113. The fluid chamber 632 is formed by an inflatable balloon 637 completely enclosing a portion of the catheter body 11 between a proximal end 634 of balloon 637 and the distal tip 136. Advantageously, balloon 637 is fixed to catheter body 11 at the proximal end 634 and at tip 136. As shown in FIGS. 22A-22B, when inflated, the balloon 637 advantageously comprises a proximal portion 635 adjacent proximal end 634 in which the balloon 637 has a circumference and/or cross section (considered perpendicular on axis 103) which increases towards the tip 136. The proximal portion 635 may extend from the proximal end 634 until an apex portion 638 where the circumference and/or cross section of balloon 637 may be maximal. Balloon 637 further comprises a distal portion 636 adjacent the tip 136 in which the circumference and/or cross section of the balloon 637 decreases towards the tip 136. The distal portion 636 may extend from the apex portion 638 to the tip 136. By way of example, the distance between proximal end 634 and tip 136 (height of balloon 637) may be from 8 mm to 50 mm, advantageously from 10 mm to 30 mm. The apex portion 638 may be located about halfway between proximal end 634 and tip 136. The maximum diameter of balloon 637, e.g., at apex portion 638, may range from 8 mm to 35 mm, advantageously from 10 mm to 20 mm when inflated. The outer diameter of catheter body 11, at least in the portion between proximal end 634 and tip 136 may be from 2.5 mm to 6 mm, advantageously from 3 mm to 5 mm.

The balloon 637 comprises one or a plurality of through holes 633 arranged on a circumference of the balloon. Through holes 633 form the outlet of delivery system 13. Through holes 633 are arranged at one or more positions along the longitudinal axis 103 advantageously located at a proximal side compared to the position of the through holes 113 of the delivery lumen. More particularly, the through holes 633 can be arranged in the proximal portion 635, and the through holes 113 can be arranged in the distal portion 636 of balloon 637.

The delivery system of FIGS. 22A-2B can be used in assemblies as described herein. When a fluid path of carrier fluid is opened, e.g., by operating switch valve 6 as described in relation to FIG. 14, a pressure in fluid chamber 632 increases thereby inflating balloon 637 and opening the through holes 633 to create a flow from supply system 12 to delivery system 13. An appropriate fluid/agent mixture is prepared in supply system 12 and injected in catheter body 11 through delivery lumen 112. The forward flow of fluid/agent mixture through delivery lumen 112 egresses from the through holes 113 to enter the fluid chamber 632 in a radial direction compared to longitudinal axis 103. The fluid/agent mixture is further guided by balloon 637 with a backwards directional component compared to the forward flow of fluid through delivery lumen 112 until the mixture reaches the through holes 633, where the mixture egresses and is delivered into the body. As a result, a backwards and radially flowing agent can be delivered.

Alternatively, or in addition, through holes 633 can be arranged in the distal portion 636 of balloon 637 to provide a forward and radial delivery of the agent.

The balloon is advantageously made of a resilient or elastic material, particularly a material, which allows to expand a circumference and/or cross section of balloon 637 when a pressure in fluid chamber 632 is increased. Examples of suitable materials are polyurethanes and silicones. Advantageously, the diameter of the through holes 633 increases when balloon 637 is inflated, while significantly decreasing when pressure in fluid chamber 632 is removed. By way of example, the through holes 633 can have a diameter from 0.2 mm to 1.5 mm, advantageously from 0.5 mm to 1 mm when balloon 637 is inflated. When deflated, the diameter of through holes 633 can decrease to 70% of their inflated diameter or less, advantageously to 55% or less. The through holes 633 can therefore act as pressure-sensitive valves which allow fluid/agent mixture to egress when a pressure in fluid chamber is increased, while substantially reducing clogging and possible ingress of bodily fluids into fluid chamber 632 when balloon 637 is not inflated, e.g., during insertion of catheter body 11 and delivery system 13 into the body.

The delivery system 13 as shown in FIGS. 22A-22B advantageously has rotational symmetry about axis 103, e.g., symmetry with respect to a rotation over an angle of 180°, 120°, 90°, 45° or 30°.

Catheter body 11 can comprise a generally atraumatic tip 639 at distal end 102, distally past the tip 136 of delivery lumen 112. The tip 639 may be highly flexible and have a decreased diameter compared to the diameter of catheter body 11 to facilitate navigation of the catheter through the body, or the tip may be ball-shaped. Alternatively, tip 136 can form the distal end 102 of the catheter body 11.

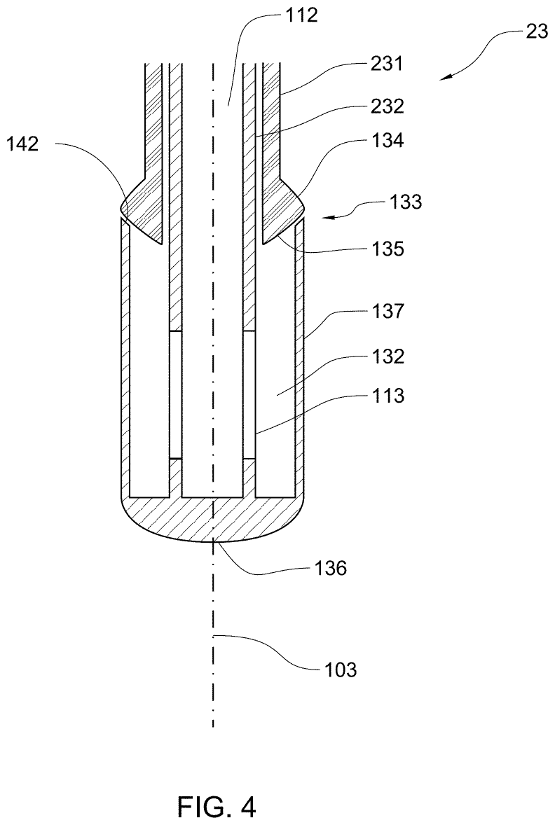

Yet alternatively, referring to FIG. 4, a valve can be created by forming the delivery system out of two parts which are moveable relative to each other in order to open and close the outlet 133 and/or the (first) outlet 113. Delivery system 23 differs from delivery system 13 in that the fluid deflecting member 134 is fixedly attached to an outer sheath 231 that may extend until the proximal end 101. Lumen 112 is provided by an inner tube 232 which is slidingly arranged in the outer sheath 231. Fluid chamber 132 is fixedly attached to inner tube 232. By sliding the inner tube 232 relative to the outer sheath 231, e.g., until an outer wall 137 of fluid chamber 132 (i.e., edge 142 of the outlet 133) abuts against the fluid deflecting member 134, the outlet 133 can be closed. Sliding in the opposite direction opens the outlet 133 while the fluid deflecting member 134 acts as fluid deflector directing fluid discharged from the fluid chamber 132 along the impingement surface 135 in a radial direction. The fluid deflecting system in delivery system 23 is otherwise identical to fluid delivery system 13. It will be convenient to note that the outer wall 137 can be made resilient and/or flexible and configured to bulge or detach from the fluid deflecting member 134. As a result, a pressure sensitive valve is obtained as with the other examples described herein.

It can be contemplated to provide a third outlet at the tip 136 in the lumen 112 or fluid chamber 132, e.g., with a spray nozzle, opening in the distal direction. In such a case, the delivery system would allow both forward and backward spraying of the medicinal agent simultaneously. The third outlet can be provided with a valve system as described with the embodiments of FIGS. 15-19.

The delivery system 13, in particular the lumen 112 and the fluid chamber 132 can be made of flexible materials, as this would ease guiding of the catheter through the body lumen or cavities. Alternatively, or in addition, the chamber 132 can be made of an inflatable material, configured to inflate due to the pressure exerted by the fluid discharged from outlet 113.

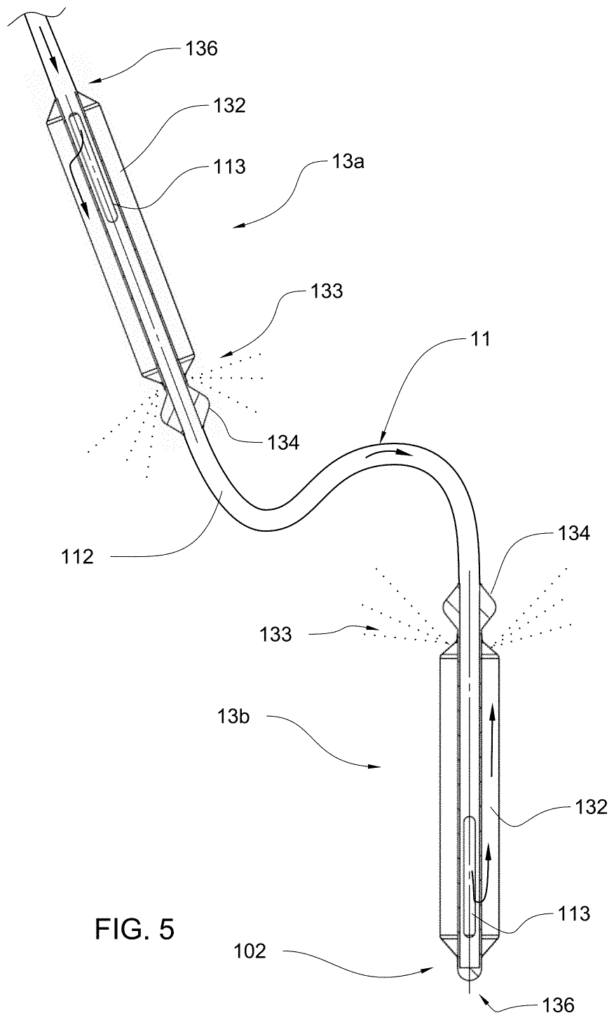

The catheter can comprise a plurality of delivery systems such as systems 13 or 23. These delivery systems may be positioned at axially distinct locations and can be coupled to a same delivery lumen 112, or to separate delivery lumens. The plurality of delivery systems may have different fluid deflection angles and/or be configured to spray in different directions. Referring to FIG. 5, the catheter can comprise two delivery systems 13a, 13b attached to the catheter body 11 at axially distinct locations, e.g., spaced apart from one another along the longitudinal axis of the catheter body 11. Both delivery systems 13a, 13b are fluidly coupled to the delivery lumen 112 to receive an agent/fluid mixture. A first delivery system 13a is arranged proximally of the second delivery system 13b. The second delivery system 13b can be identical to the ones described previously. The first delivery system 13a is connected to the lumen 112 in the inverse sense, e.g., with tip 136 arranged at the proximal side and the outlet 133 arranged at the distal side of the delivery system. The first delivery system 13a is open at the distal tip 136 where it is in fluid connection with the proximal part of lumen 112. At the distal side, lumen 112 crosses the first delivery system 13a and continues further to the second delivery system 13b. The flow of a fluid/agent mixture is indicated by the arrows, with the first delivery system 13a arranged to spray in forward direction (same direction as the flow in lumen 112) and the second delivery system 13b arranged to spray in backward direction compared to the flow direction in lumen 112, as indicated by the dotted lines in FIG. 5. Such a configuration advantageously allows for delivering the fluid/agent mixture to a larger area at once, covering the space between the first and second delivery systems.

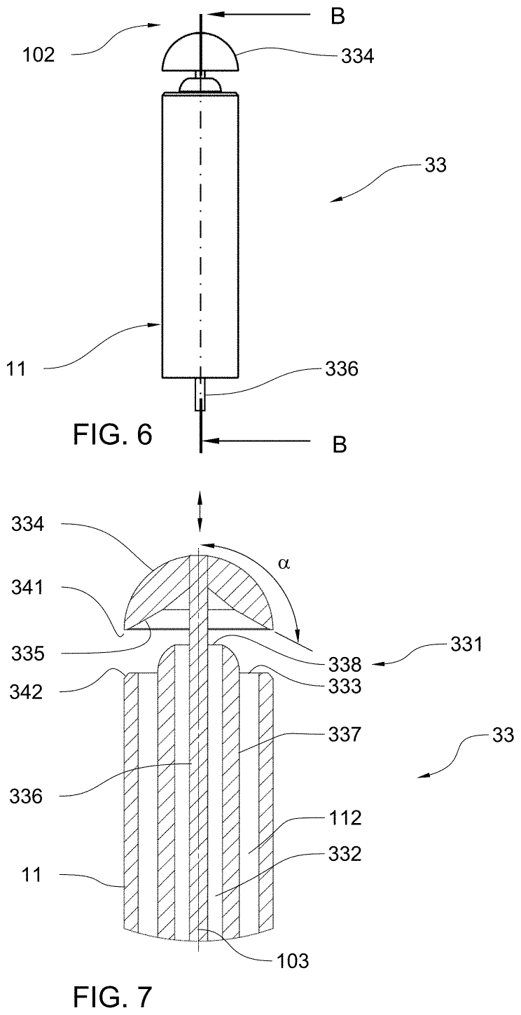

Referring to FIGS. 6 and 7, an alternative delivery system 33 comprises a fluid deflecting system 331 that is somewhat different compared to the fluid deflecting system 131 of FIGS. 2-4. Delivery system 331 comprises delivery lumen 112 formed in catheter body 11. An inner lumen 332 is arranged inside delivery lumen 112. Inner lumen 332 can be coaxial with delivery lumen 112, and delivery lumen 112 can completely surround inner lumen 332. Inner lumen 332 is formed by a fluid supply duct 337 which advantageously separates inner lumen 332 from delivery lumen 112. Alternatively, a plurality of delivery lumens can be arranged at a circumference of the fluid supply duct 337, e.g., at separate angular positions.

Delivery lumen 112 extends in the distal direction until outlet 333, from where a fluid/agent mixture is discharged. Fluid supply duct 337 advantageously extends in the distal direction until past the outlet 333 and discharges at fluid outlet 338. Fluid outlet 338 is advantageously arranged at a position along the longitudinal axis 103 which is more distal than the position of outlet 333. The fluid deflecting system further comprises a fluid deflecting member 334 which has an inner surface acting as a fluid impingement surface 335 which extends over (and covers) at least the fluid outlet 338, and preferably also over outlet 333. Fluid deflecting member 334 is advantageously formed as dome-shaped cap to cover the fluid outlet 338 (fluid supply duct 337) and preferably also outlet 333 (catheter body 11). The inner surface of the dome-shaped cap is in this case formed by the impingement surface 335. The impingement surface 335 hence faces at least the fluid outlet 338.

A push rod 336 is attached to the fluid deflecting member 334. Push rod 336 extends along the catheter body 11, e.g., within the inner lumen 332, until the proximal end 102, from where it can be actuated to move the fluid deflecting member 334 relative to the catheter body 11. Advantageously, a peripheral rim 341 of fluid deflecting member (cap) 334 conforms to a peripheral edge of the catheter body 11, in particular to a circumferential edge 342 of the outlet 333. Advantageously, a diameter of peripheral rim 341 of fluid deflecting member is substantially equal to an outer diameter of the catheter body. By so doing, the fluid deflecting member 334 can be pulled by push rod 336 to close the outlet 333 in order to prevent ingress of bodily fluids or moisture inside the delivery lumen 112, which may deteriorate the agent that is to be delivered. At the treatment site, the rod 336 can be pushed in distal direction relative to the catheter body 11 to open the outlet 333. As a result, the fluid deflecting member 334 and push rod 336 can act as a valve system.

In use, a fluid, which can be a gas or a liquid, is supplied through the inner lumen 332 and discharged from fluid outlet 338. As the impingement surface 335 is arranged opposite the fluid outlet 338, the fluid impinges on the surface 335 and is deflected radially outward, thereby passing the outlet 333, where it intersects with the agent, or fluid/agent mixture that is discharged through outlet 333. The fluid from outlet 338 and deflected by surface 335 will therefore entrain any fluid/agent mixture that is discharged from the outlet 333 into a radially outward direction. As a result, the agent is sprayed in a radial and backwards direction due to the orientation of the impingement surface 335.

Advantageously, only a fluid, such as a gas, and no agent is supplied through inner lumen. The agent is advantageously supplied only through the delivery lumen 112. One advantage of the inner lumen 332 is that it improves evacuation of a powdery agent that is discharged from the delivery lumen at outlet 333, avoiding the powder to heap up between the outlet 333 and the impingement surface. This is obtained with a delivery system 33 having very compact dimensions, e.g., dimensions which do not exceed those of the catheter body 11. It will therefore be convenient to note that the delivery system 33 advantageously has an outer cylindrical shape without protrusion, and with a substantially arcuate, spherical or at least atraumatic cap at the distal end.

Impingement surface 335 is appropriately inclined relative to the longitudinal axis 103 to obtain a deflection of the fluid which is radially outward and in a backward direction relative to the fluid flow in lumens 112 and 332. Advantageously impingement surface 335 is inclined at an angle α as indicated hereinabove and measured as from a distal (forward) side of the longitudinal axis 103.

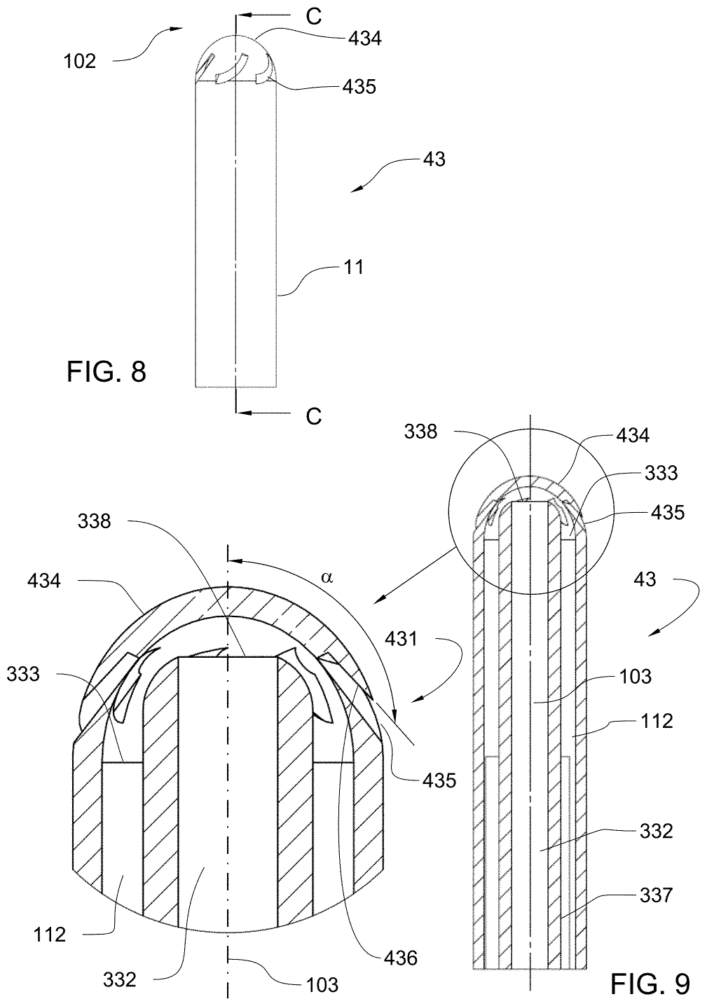

Referring to FIGS. 8 and 9, yet another delivery system 43 according to aspects of the present disclosure differs from delivery system 33 in that the fluid deflecting system 431 comprises a fluid deflecting member 434 which is fixedly attached to the catheter body 11. Fluid deflecting member 434 forms a dome-shaped cap that covers the delivery lumen 112. Both delivery lumen 112 and inner lumen 332 are advantageously cylindrical shaped and concentric.

A plurality of exit holes 435 are arranged through the fluid deflecting member 434, advantageously at circumferential locations around the member. Exit holes 435 are in fluid communication with outlet 333 of delivery lumen 112 and fluid outlet 338 of inner lumen 332. Advantageously, the exit holes 435 are positioned at an intermediate position along the longitudinal axis 103 between outlet 333 and fluid outlet 338, and they may extend distally past the outlet 338. Advantageously, the exit holes 435 are arranged at a radial position substantially corresponding with the outlet 333, and may be arranged radially past the outlet 333.

The exit holes 435 comprise a fluid deflection surface 436 forming an inner wall of the exit holes. The fluid deflection surface is particularly formed by a distal portion of the inner wall of the exit holes. The fluid deflection surface 436 is inclined to deflect fluid discharged from fluid outlet 338 to the exit holes 435. By so doing, the flow of fluid crosses the fluid/agent mixture discharged from outlet 333 and entrains it towards the exit holes 435, from where the agent will be sprayed in a radially outward direction, and backwards, towards the proximal side. An angle of inclination a of the fluid deflection surface 436 is advantageously as indicated above, in relation to the embodiments described above.

Fluid discharged from fluid outlet 338 is deflected by the dome-shaped inner wall of the fluid deflecting member 434 towards the exit holes 435. The fluid will entrain the fluid/agent mixture supplied by delivery lumen 112 along the inner wall of member 434 towards the exit holes 435 from where it exits the delivery system 43 in a radially outward and backward direction compared to the proximal-distal direction. The fluid deflecting member 434 may further comprise one or more holes that are configured to spray a fluid/agent mixture in a forward direction. These holes may e.g., be interleaved with the exit holes 435.

The delivery system 43 does not comprise a valve, even though a pressure-sensitive valve closing the exit holes 435 may be contemplated. Entraining fluid, such as carbon dioxide gas can be continuously supplied through the inner lumen 332 during insertion and manipulation of the catheter inside the body in order to prevent ingress of bodily fluids of moisture through the exit holes 435. This delivery system 43 may be used along with a suction system, e.g., a suction pipe, in order to evacuate the supplied entraining fluid from the body and prevent damage to the body lumen or cavity by excessive insufflation.

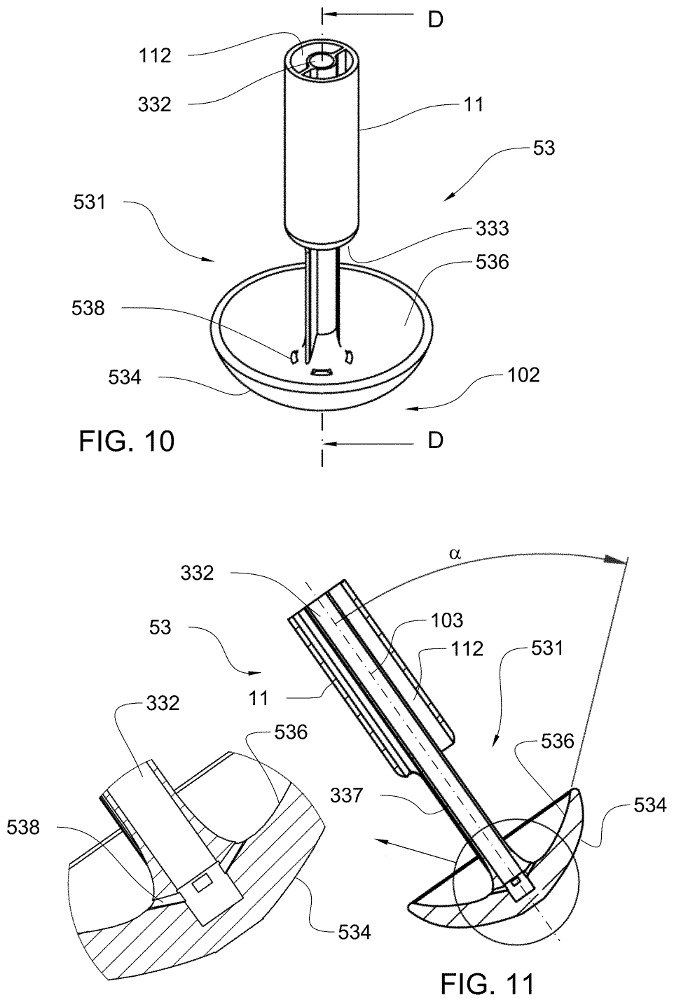

Referring to FIGS. 10 and 11, an alternative delivery system 53 is shown that can be used in catheters 10 according to aspects of the present disclosure. Delivery system 53 differs from delivery system 43 in that the fluid deflection system 531 comprises an advantageously dome-shaped fluid deflecting member 534. An advantageously concave surface 536 of fluid deflecting member 534 is facing the outlet 333 of the delivery lumen 112 at a spaced apart distance. Advantageously, the concave surface 536 extends radially beyond the catheter body 11. That is, a diameter of an outer rim of the fluid deflecting member 534 is advantageously larger, e.g., at least 1.2 times larger, than an outer diameter of the catheter body 11, or than a diameter of the delivery lumen 112. The fluid supply lumen 332 is provided in an inner duct 337, just like in the embodiment of FIGS. 6-7 and 8-9. The inner duct 337 extends in a distal direction past the outlet 333 of delivery lumen 112. Inner duct 337 may be coaxial and advantageously concentric with catheter body 11 which defines the delivery lumen 112.

The fluid deflecting member 534 is advantageously attached at a distal end of the inner duct 337. The fluid supply lumen 332 advantageously extends until the fluid deflecting member 534. The fluid supply lumen 332 advantageously comprises a plurality of outlet channels 538 extending substantially radially outwards relative to the longitudinal axis 103 to fluidly communicate with the concave surface 536. The outlet channels 538 may open flush with concave surface 536. Delivery system 53 may have rotational symmetry about the longitudinal axis 103, in particular a rotational symmetry with respect to a rotation by an angle of 180°, 120°, 90° or possibly 45°. With such a configuration, fluid discharged from outlet channels 538 and fluid discharged from outlet 333 will cross in a space between the outlet 333 and the surface 536.

In use, a fluid such as carbon dioxide gas is insufflated through the fluid supply lumen 332. The fluid exits the fluid supply lumen through the outlet channels 538 which convey it over the concave surface 536. Concave surface 536 advantageously directs the fluid in a radial and backwards (proximal) direction. The concave surface 536, in particular an outer edge portion thereof where fluid detaches from the concave surface, is advantageously inclined with respect to the longitudinal axis at an angle α as indicated hereinabove. Simultaneously, the agent is transported through the delivery lumen 112, e.g., with a carrying fluid as an agent/fluid mixture. The carrying fluid may be the same fluid as the fluid through fluid supply lumen 332. As the agent is discharged from outlet 333 in a direction towards the concave surface 536, it is advantageously entrained by the fluid exiting the outlet channels 538 and is deflected in a radial and backwards direction to reach treatment zones that are arranged at the rear of the delivery system 53. Furthermore, the outlet channels 538 advantageously remove any agent that may impinge on concave surface 536.

Since only fluid passes through the outlet channels 538, these can be made suitably small as there is a reduced risk of clogging. As with the embodiments above, it can be contemplated to provide a valve (not shown) at the outlet 333 to prevent ingress of bodily fluids into the delivery lumen 112 during insertion and manipulation of the catheter inside the body. It will however be convenient to note that a valve may be omitted since the lumen 112 is advantageously straight at the distal end with the aperture of outlet 333 being in a plane substantially orthogonal to the longitudinal axis 103. Hence, in the delivery system 53, the deflection of the agent is made to occur between the outlet 333 and the concave surface 536, and hence outside of the system, such that risk of clogging of the agent, e.g., in case of powdery agents, is minimized.

The fluid deflecting member 534 can be made of a flexible or resilient material. It can be contemplated to make the member deployable, so as to reduce bulkiness during insertion through the body orifice.

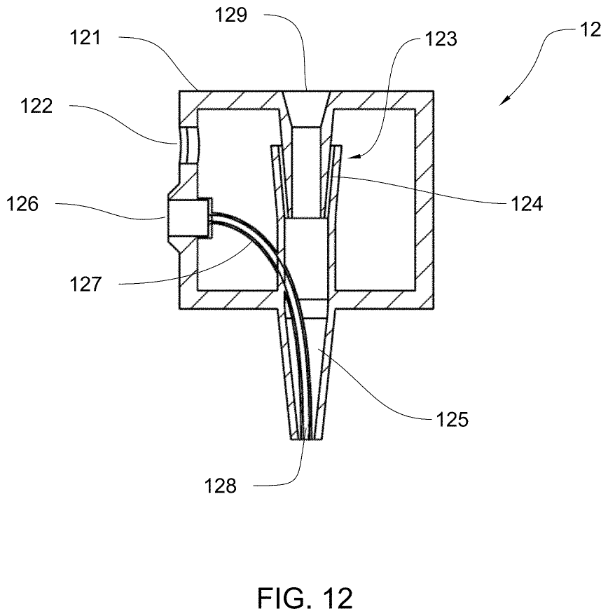

Any of the above delivery systems are advantageously used in combination with a supply system 12 as represented in FIG. 12. Supply system 12 is advantageously configured to prepare a mixture of agent and carrying fluid in suitable proportions. The fluid is advantageously a gas, such as carbon dioxide, but may alternatively be a liquid. The agent is advantageously a powder, but can alternatively be a liquid, in which case an aerosol can be obtained, or even a gas.

Supply system 12 as represented in FIG. 12 is advantageously suited for preparation of a mixture of gas and a powder agent. It comprises a fluid chamber 121 having a fluid (gas) input port 122. A Venturi mixing device 123, i.e. a mixing device utilizing the Venturi effect for aspirating agent, comprises a constriction 124, referred to as Venturi channel, arranged at an outlet of the fluid chamber 121. The constriction 124 is arranged concentrically about an agent supply tube 129. The Venturi channel 124 is in fluid communication with the fluid chamber 121 at its inlet. Both the Venturi channel 124 and the agent supply tube 129 are in fluid communication with the output port 125 that delivers a mixture of gas and agent supplied from the input port 122 and the supply tube 129, respectively. The mixture is obtained by the Venturi effect created by a flow of the carrier fluid through the constriction 124, which aspirates the agent out of supply tube 129. The Venturi mixing device 123 is advantageously housed within the fluid chamber 121. The output port 125 is advantageously connected to the delivery lumen 112 of catheter body 11, e.g., through connector 111 (see FIG. 1).

One advantage of a Venturi mixing device is that it is a self-regulating system. When the flow of carrier fluid is stopped, e.g., by operating the switch valve 6 in FIG. 14 or due to a clog occurring in the delivery lumen, the flow of gas through the Venturi channel is stopped, which will automatically stop the aspiration of agent from the supply tube 129. This prevents the delivery lumen to become clogged with powder. It has been found that a Venturi mixing device prevents clogging of the delivery system 13 when a valve 14 is provided to prevent ingress of moisture and bodily fluids. Furthermore, when valve 14 is a pressure sensitive valve, the Venturi mixing device as described herein allows to start delivering agent only when the valve opens since this will entrain a flow of the carrier fluid. The Venturi mixing device therefore prevents that agent would be aspirated in the delivery lumen when pressure is building up and before the valve opens.

It has been found that the above synergy is improved with the Venturi designs as described herein. In particular, when the Venturi channel is provided around and advantageously concentric to the input port 129 of the agent. Advantageously, the Venturi channel 124 has an axis at an angle from 0° to 45° of an axis of the output port 125.

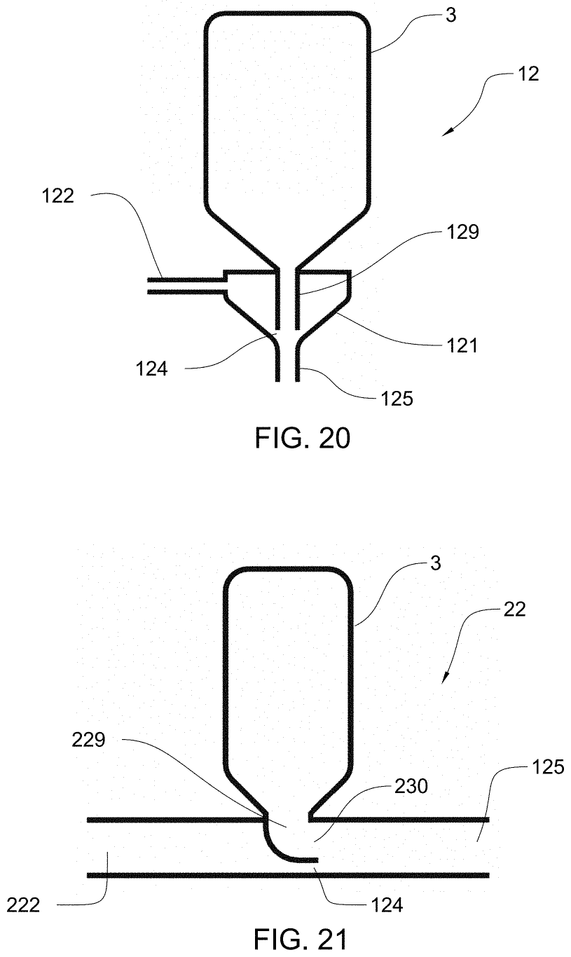

Referring to FIG. 20, another beneficial configuration for the supply system 12 is shown, which is similar to the supply system of FIG. 12. In the supply system of FIG. 20, as with the supply system of FIG. 12, the supply tube 129 of the agent and the output port 125 for the mixture are advantageously aligned. The constriction 124 for creating the Venturi effect in FIG. 20 is somewhat shorter but still concentric with the supply tube 129.

An alternative beneficial configuration of the supply system is shown in FIG. 21. Supply system 22 comprises a supply tube 229 fluidly coupled to reservoir 3 of the agent and an input duct 222 for the carrier fluid, which may be fluidly coupled to the source 1. Input duct 222 is aligned with the output port 125. The supply tube 229 comprises an egress port 230 which furthermore has an axis parallel to the axes of the input duct 222 and the output port 125. The axis of egress port 230 is offset from the axis of the output port 125 and/or the input duct 222. A constriction 124 for the Venturi effect is created by the egress port 230 protruding into the input duct 222, and therefore the constriction 124 is not concentric with the egress port 230.

Referring again to FIG. 12, for the embodiments of FIGS. 6-7, 8-9, 10-11, and 18, the supply system 12 and/or 22 can additionally comprise a secondary fluid (gas) supply port 126, advantageously arranged in a wall of the fluid chamber 121. The secondary fluid supply port 126 is in fluid communication with a secondary fluid output port 128 through a duct 127 that advantageously is fluidly isolated from the fluid chamber 121. Fluid output port 128 is configured for being connected to the fluid supply lumen 332 through a suitable connection system as known in the art, e.g., a Luer lock.

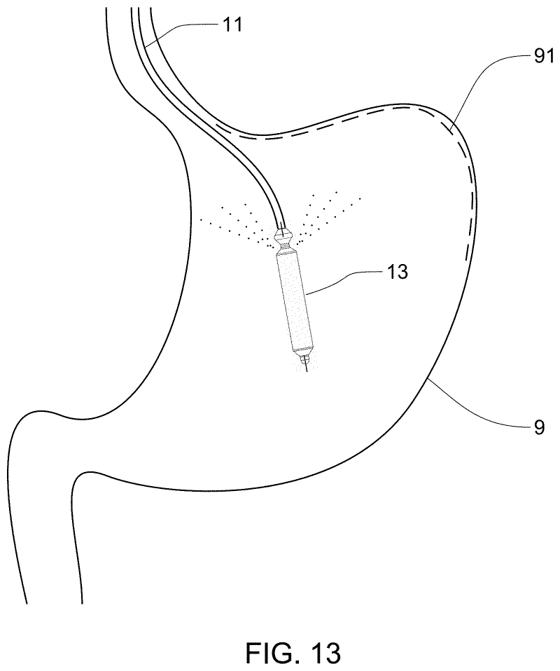

Referring to FIG. 13, devices as described herein are particularly advantageous for treatment of gastrointestinal bleeding through delivery of a hemostatic agent. To do so, the delivery system attached at the distal end of the catheter body is advantageously introduced in the body through the nasal cavity into the GI tract. The catheter body can be further introduced until it reaches the treatment site, e.g., the stomach 9. The position of the catheter body inside the body can be adjusted according to different methods. In one example, graduations are provided on the catheter body to indicate what length has been inserted allowing the practitioner to adjust the depth of the insertion according to the corpulence of the patient. For treatment in the stomach, the output of the (most distal) delivery system of the catheter should be inserted until about 55 cm from the dental arcade for tall persons and a minimum of about 45 cm for smaller persons. Alternatively, an inflatable balloon is attached to the catheter, which can be inflated when inside the stomach. The balloon is positioned relative to the delivery system such that, when the practitioner pulls on the catheter body until the balloon makes contact with the cardia, the catheter will be in the right position inside the upper gastro-intestinal tract. The balloon can then be deflated. Yet alternatively, gas is supplied through the supply system 12, either through the delivery lumen 112, the fluid supply lumen 332, or both and without delivery of agent. The sound of the gas flow touching the gastric wall is heard using a stethoscope, and the operator may know when the delivery system arrives at the treatment site, e.g., the stomach. Other techniques to correctly position the catheter inside the GI tract without requiring vision can be contemplated.

At this point, the agent can be supplied to supply system 12, which may prepare a fluid/agent mixture that is delivered to the delivery system 13, from where it is delivered to the bleeding site 91. As shown in FIG. 13, the bleeding site 91 may well be located at the rear of the delivery system, e.g., the bleeding site may be surrounding the cardia, and catheters as described herein are particularly suitable to deliver such agents at such sites which are difficult to reach and without needing to bend the catheter, and without requiring visual feedback. Possibly, after or at the same time, the agent is sprayed distally, e.g., from another delivery system located proximally compared to the one delivery system, e.g., in order to deliver the agent in the esophagus. Alternatively, a same delivery system can be used, which also allows to spray in forward (distal) direction. The latter step can be repeated several times or performed continuously while pulling the catheter body over several centimeters until at distance of 30 cm from the dental arcade. Finally, the catheter can be extracted in a non-traumatic manner.

The catheter body and/or delivery system are advantageously made of a biocompatible material, in particular a biocompatible polymer or metal, such as stainless steel or titanium alloys. Suitable examples of such polymers are polyurethane, polyvinyl chloride, polytetrafluoroethylene, polyamide, such as nylon, polyethylene terephthalate and polyethylene (in any of its forms e.g., as high, medium or low density). Alternatively, elastomeric biocompatible materials may be used as well, such as natural rubber, or thermoplastic elastomers.

The dimensions of the catheter body and of the delivery system are advantageously adapted to the dimensions of the natural orifice through which the catheter is introduced in the body. Advantageously, catheter body 11 has an external diameter of at most 6 mm, advantageously at most 5.5 mm, advantageously at most 5.33 mm (i.e., 16 French). When used in an instrument channel of an endoscope, the external diameter of the catheter body 11 is advantageously at most 10 French, advantageously at most 7 French, advantageously at most 5 French. The fluid deflecting member 534 of delivery system 53 is advantageously made of a flexible and resilient material, in particular an elastomeric material, and as such can be larger than the diameter of the catheter body. The catheter body can have a length from 65 to 85 cm. Alternatively, the catheter body can have a length of at least 103 cm, advantageously at least 135 cm, advantageously at least 200 cm. The resilient membrane of the valve member can be made of a silicone or polyurethane material.

### C. Illustrative Combinations and Additional Examples

This section describes additional aspects and features of INVENTION, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A. Catheter (10) for delivering an agent, comprising:
a catheter body (11) comprising a first lumen (112) extending from a proximal end (101) to a distal end (102) and defining a longitudinal axis (103),
a first outlet (113, 333) adjacent to the distal end and in fluid communication with the first lumen (112),
wherein the catheter comprises fluid deflecting means (132, 331, 431, 531) configured to deflect a flow of the agent discharged from the first outlet (113, 333) in a direction having a backwards component relative to the flow in the first lumen, the fluid deflecting means comprising a fluid deflecting surface (137, 335, 436, 536) arranged opposite the first outlet (113, 333).

B. Catheter of clause A, wherein the fluid deflecting surface is inclined with respect to the longitudinal axis at an angle from 95° to 180° measured from a distal side of the longitudinal axis (103).

C. Catheter of clause A or B, comprising a valve (14) configured to close the first lumen (112) from ambient environment.

D. Catheter of any one of the preceding clauses A-C, wherein the fluid deflecting means comprises a fluid channel (132) having a second outlet (133) arranged at a proximal side compared to the first outlet (113), wherein the fluid channel extends parallel to the first lumen between the first outlet (113) and the second outlet (133).

E. Catheter of clause D, wherein the first outlet is arranged in a side wall of the first lumen and wherein a side wall (137) of the fluid channel (132) forms the fluid deflecting surface.

F. Catheter of clause D or E, wherein the fluid deflecting means comprises a second fluid deflecting surface (135) arranged opposite the second outlet (133), wherein the second fluid deflecting surface (135) is arranged to impart a direction change of the flow of the agent discharged from the second outlet (133) compared to the flow of the agent through the fluid channel (132).

G. Catheter of clause F, wherein the fluid deflecting means (131) comprises a radial protrusion (134) attached to the catheter body (11), the second fluid deflecting surface (135) being a surface of the radial protrusion.

H. Catheter of any one of the preceding clauses A-G, wherein the fluid deflecting means (331, 431, 531) comprises a second lumen (332) extending from the proximal end (101) to the distal end (102) adjacent to the first lumen and a fluid deflecting member (334, 434, 534), wherein the second lumen comprises a second outlet (338, 538), wherein the fluid deflecting member is configured to deflect a fluid discharged from the second outlet (338, 538) towards an outlet (113, 333) of the first lumen, thereby intersecting a fluid path exiting the first lumen.

I. Catheter of clause H, wherein the outlet of the first lumen is the first outlet (133), wherein the fluid deflecting member is a cap (334, 434, 534) arranged opposite the second outlet (338) and spaced apart from the second outlet (338, 538).

J. Catheter of clause I, wherein the fluid deflecting surface (335, 436, 536) extends at an edge of the cap (334, 434, 534).

K. Catheter of clause I or J, wherein the cap (436, 536) is fixed to the catheter body (11).

L. Catheter of clause K, wherein the fluid deflecting surface (436) forms a portion of an inner wall of a hole (435) through the cap (434).

M. Catheter of any one of the clauses I to L, wherein the cap (534) is dome-shaped and spaced apart from the first outlet (333).

N. Catheter of any one of the clauses A to L, wherein the fluid deflecting surface is moveable between a first position wherein the fluid deflecting surface (135, 335) abuts against an edge (142, 342) of the first outlet and a second position wherein the fluid deflecting surface is spaced apart from the first outlet.

O. Assembly for delivering an agent, comprising the catheter (10) of any one of the preceding clauses A to N, and a supply system (12) comprising an output port configured for connection to the catheter body (11) at the proximal side (101), wherein the supply system comprises a Venturi mixing device (123) configured to prepare a mixture of a fluid and the agent, wherein an output of the Venturi mixing device is fluidly coupled with the first lumen (112).

P0. An assembly for delivering an agent, the assembly comprising a catheter and a supply system,
wherein the catheter comprises:
a catheter body comprising a first lumen extending from a proximal end to a distal end and defining a longitudinal axis, and
a first outlet adjacent to the distal end and in fluid communication with the first lumen;
wherein the supply system is arranged at a proximal end of the first lumen and comprises a first inlet port for a carrier fluid, a second inlet port for the agent and an output port in fluid communication with the first and second inlet ports and with the first lumen;
wherein the supply system comprises a fluid path from the first inlet port to the output port, the fluid path comprising a constriction in vicinity of the second inlet port configured to aspirate the agent from the second inlet port by a Venturi effect; and
wherein the catheter further comprises a pressure-sensitive valve arranged adjacent to the first outlet and in fluid communication with the first lumen.

P1. The assembly of P0, wherein the pressure-sensitive valve comprises a valve seat and a pressure-sensitive valve member, wherein the pressure sensitive valve member is a resilient membrane attached circumferentially to the catheter body and surrounding the valve seat.

P2. The assembly of P1, wherein a portion of the resilient membrane is shaped as a sleeve sealing against the catheter body.

P3. The assembly of P2, wherein the sleeve is substantially cylindrical.

P4. The assembly of P1, wherein the pressure-sensitive valve member is a resilient membrane having a through-opening, wherein the resilient membrane comprises an inner edge defining a perimeter of the through-opening, and wherein the inner edge seals against the valve seat in a closed state of the valve and is deflected from the valve seat in an open state of the valve.

P5. The assembly of P4, wherein, in a closed state of the valve, the inner edge is axially spaced apart from an attachment portion of the resilient membrane to the catheter body.

P6. The assembly of P4 or P5, wherein the resilient membrane comprises a distal portion adjacent to the inner edge, the distal portion of the resilient membrane having a shape tapering towards the valve seat.

P7. The assembly of P1, wherein the valve seat is substantially concentric to the first outlet.

P8. The assembly of P1, wherein the valve seat has a circular cross section.

P9. The assembly of P1, wherein the catheter body comprises a second lumen surrounded by the first lumen, and wherein the valve seat surrounds the second lumen.

P10. The assembly of P1, wherein the valve seat is attached to a rod extending along the catheter body, the rod configured to allow the valve seat to be axially moved relative to the catheter body.

P11. The assembly of P1, wherein the pressure-sensitive valve member is arranged having an external surface exposed to ambient.

P12. The assembly of P1, wherein the second inlet port and the output port have parallel axes.

P13. The assembly of P12, wherein the constriction has an axis at an angle from 0° to 45° of an axis of the output port.

P14. The assembly of P1, wherein the constriction and the second inlet port are concentric.

P15. The assembly of P14, wherein the constriction completely surrounds the output port.

Q0. An assembly for delivering an agent, the assembly comprising a catheter and a supply system,
wherein the catheter comprises:
   a catheter body comprising a first lumen extending from a proximal end to a distal end and defining a longitudinal axis, and
   a first outlet adjacent to the distal end and in fluid communication with the first lumen;
   wherein the supply system is arranged at a proximal end of the first lumen and comprises a first inlet port for a carrier fluid, a second inlet port for the agent and an output port in fluid communication with the first and second inlet ports and with the first lumen;
   wherein the supply system comprises a fluid path from the first inlet port to the output port, the fluid path comprising a constriction in vicinity of the second inlet port configured to aspirate the agent from the second inlet port by a Venturi effect;
   wherein the catheter comprises a fluid deflecting system in fluid communication with the first outlet, wherein the fluid deflecting system is configured to discharge a flow of the agent from the first outlet in a direction having a backwards component relative to a flow in the first lumen; and
   wherein the fluid deflecting system comprises a fluid chamber in fluid communication with an egress port of the first lumen, wherein the fluid chamber comprises a surface arranged opposite the egress port and configured to deflect a flow discharged from the egress port towards the first outlet.

Q1. The assembly of Q0, wherein the egress port is arranged in a side wall of the first lumen and wherein the surface is a side wall of the fluid chamber.

Q2. The assembly of Q0 or Q1, wherein the egress port is arranged between the first outlet and the distal end along the longitudinal axis.

Q3. The assembly of any one of paragraphs Q0 through Q2, wherein the fluid chamber is inflatable.

R0. An assembly for delivering an agent, comprising a catheter (10) and a supply system (12), wherein the catheter (10) comprises:
a catheter body (11) comprising a first lumen (112) extending from a proximal end (101) to a distal end (102) and defining a longitudinal axis (103), and
a first outlet (133) adjacent to the distal end and in fluid communication with the first lumen (112),
wherein the supply system (12) is arranged at a proximal end of the first lumen and comprises a first inlet port (122) for a carrier fluid, a second inlet port (129) for the agent and an output port (125) in fluid communication with the first and second inlet ports and with the first lumen (112),
wherein the supply system (12) comprises a fluid path from the first inlet port (122) to the output port (125), the fluid path comprising a constriction (124) in vicinity of the second inlet port (129) configured to aspirate the agent from the second inlet port by the Venturi effect,
characterized in that the catheter (10) comprises a valve (14) arranged adjacent to the first outlet (133) and in fluid communication with the first lumen (112), wherein the valve comprises a valve seat (140) and a pressure-sensitive valve member (141).

R1. The assembly of R0, wherein the pressure sensitive valve member is a resilient membrane (141) attached circumferentially to the catheter body (11) and surrounding the valve seat (140).

R2. The assembly of R1, wherein a portion of the resilient membrane (141) is shaped as a sleeve (143) sealing against the catheter body (11).

R3. The assembly of R2, wherein the sleeve (143) is substantially cylindrical.

R4. The assembly of any one of paragraphs R0 through R3, wherein the pressure-sensitive valve member is a resilient membrane (141) having a through-opening (149), wherein the resilient membrane comprises an inner edge (145) defining a perimeter of the through-opening (149), wherein the inner edge (145) seals against the valve seat (140) in a closed state of the valve and is deflected from the valve seat in an open state of the valve.

R5. The assembly of R4, wherein, in a closed state of the valve, the inner edge (145) is axially spaced apart from an attachment portion of the resilient membrane to the catheter body.

R6. The assembly of R4 or R5, wherein the resilient membrane (141) comprises a distal portion (144) adjacent to the inner edge (145), the distal portion of the resilient membrane having a shape tapering towards the valve seat (140).

R7. The assembly of any one of paragraphs R0 through R6, wherein the valve seat (140) is substantially concentric to the first outlet (133).

R8. The assembly of any one of paragraphs R0 through R7, wherein the valve seat (140) has circular cross section.

R9. The assembly of any one of paragraphs R0 through R8, wherein the catheter body (11) comprises a second lumen (212) surrounded by the first lumen, wherein the valve seat (140) surrounds the second lumen.

R10. The assembly of any one of paragraphs R0 through R9, wherein the valve seat (140) is attached to a rod (246) extending along the catheter body, the rod allowing the valve seat to be axially moved relative to the catheter body.

R11. The assembly of any one of paragraphs R0 through R10, wherein the pressure-sensitive valve member is arranged having an external surface exposed to ambient.

R12. The assembly of any one of paragraphs R0 through R11, wherein the second inlet port (129, 230) and the output port (125) have parallel axes.

R13. The assembly of R12, wherein the constriction (124) has an axis at an angle between 0° and 45° of an axis of the outlet port.

R14. The assembly of any one of paragraphs R0 through R13, wherein the constriction (124) and the second inlet port (129, 230) are concentric.

R15. The assembly of R14, wherein the constriction (124) completely surrounds the output port (125).

R16. The assembly of any one of paragraphs R0 through R15, wherein the catheter comprises fluid deflecting means (131) configured to deflect a flow of the agent discharged from the first lumen (112) in a direction having a backwards component relative to the flow in the first lumen, wherein the fluid deflecting means comprises a fluid channel (132) in fluid communication with an egress port (113) of the first lumen (112), wherein the fluid channel extends parallel to the first lumen (112) between the egress port (113) and the first outlet (133), wherein the first outlet (133) is arranged at a proximal side compared to the egress port (113), wherein the fluid deflecting means comprises a first fluid deflecting surface (137) arranged opposite the egress port (113) for deflecting a flow discharged from the egress port (113) towards the first outlet (133), wherein the fluid deflecting means comprises a second fluid deflecting surface (135) arranged opposite the first outlet (133) and configured to impart a direction change to the flow discharged from the first outlet (133) compared to the flow through the fluid channel (132).

R17. The assembly of R16, wherein the egress port (113) is arranged in a side wall of the first lumen (112) and wherein a side wall (137) of the fluid channel (132) forms the fluid deflecting surface.

R18. The assembly of R16 or R17, wherein the fluid deflecting means (131) comprises a radial protrusion (134) attached to the catheter body (11), the second fluid deflecting surface (135) being a surface of the radial protrusion.

R19. The assembly of any one of R16 through R18, wherein a wall of the first lumen (112) forms the valve seat (140).

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. An assembly for delivering an agent, the assembly comprising a catheter and a supply system,
wherein the catheter comprises:
a catheter body comprising a first lumen extending from a proximal end to a distal end and defining a longitudinal axis, and
a first outlet adjacent to the distal end and in fluid communication with the first lumen;
wherein the supply system is arranged at a proximal end of the first lumen and comprises a first inlet port for a carrier fluid, a second inlet port for the agent and an output port in fluid communication with the first and second inlet ports and with the first lumen;
wherein the supply system comprises a fluid path from the first inlet port to the output port, the fluid path comprising a constriction in vicinity of the second inlet port configured to aspirate the agent from the second inlet port by a Venturi effect; and
wherein the catheter further comprises a pressure-sensitive valve arranged adjacent to the first outlet and in fluid communication with the first lumen.

2. The assembly of claim 1, wherein the pressure-sensitive valve comprises a valve seat and a pressure-sensitive valve member, wherein the pressure sensitive valve member is a resilient membrane attached circumferentially to the catheter body and surrounding the valve seat.

3. The assembly of claim 2, wherein a portion of the resilient membrane is shaped as a sleeve sealing against the catheter body.

4. The assembly of claim 3, wherein the sleeve is substantially cylindrical.

5. The assembly of claim 2, wherein the pressure-sensitive valve member is a resilient membrane having a through-opening, wherein the resilient membrane comprises an inner edge defining a perimeter of the through-opening, and wherein the inner edge seals against the valve seat in a closed state of the valve and is deflected from the valve seat in an open state of the valve.

6. The assembly of claim 5, wherein, in a closed state of the valve, the inner edge is axially spaced apart from an attachment portion of the resilient membrane to the catheter body.

7. The assembly of claim 5, wherein the resilient membrane comprises a distal portion adjacent to the inner edge, the distal portion of the resilient membrane having a shape tapering towards the valve seat.

8. The assembly of claim 2, wherein the valve seat is substantially concentric to the first outlet.

9. The assembly of claim 2, wherein the valve seat has a circular cross section.

10. The assembly of claim 2, wherein the catheter body comprises a second lumen surrounded by the first lumen, and wherein the valve seat surrounds the second lumen.

11. The assembly of claim 2, wherein the valve seat is attached to a rod extending along the catheter body, the rod configured to allow the valve seat to be axially moved relative to the catheter body.

12. The assembly of claim 2, wherein the pressure-sensitive valve member is arranged having an external surface exposed to ambient.

13. The assembly of claim 2, wherein the second inlet port and the output port have parallel axes.

14. The assembly of claim 13, wherein the constriction has an axis at an angle from 0° to 45° of an axis of the output port.

15. The assembly of claim 2, wherein the constriction and the second inlet port are concentric.

16. The assembly of claim 15, wherein the constriction completely surrounds the output port.

17. An assembly for delivering an agent, the assembly comprising a catheter and a supply system,
wherein the catheter comprises:
a catheter body comprising a first lumen extending from a proximal end to a distal end and defining a longitudinal axis, and
a first outlet adjacent to the distal end and in fluid communication with the first lumen;
wherein the supply system is arranged at a proximal end of the first lumen and comprises a first inlet port for a carrier fluid, a second inlet port for the agent and an output port in fluid communication with the first and second inlet ports and with the first lumen;
wherein the supply system comprises a fluid path from the first inlet port to the output port, the fluid path comprising a constriction in vicinity of the second inlet port configured to aspirate the agent from the second inlet port by a Venturi effect;
wherein the catheter comprises a fluid deflecting system in fluid communication with the first outlet, wherein the fluid deflecting system is configured to discharge a flow of the agent from the first outlet in a direction having a backwards component relative to a flow in the first lumen; and
wherein the fluid deflecting system comprises a fluid chamber in fluid communication with an egress port of the first lumen, wherein the fluid chamber comprises a surface arranged opposite the egress port and configured to deflect a flow discharged from the egress port towards the first outlet.

18. The assembly of claim 17, wherein the egress port is arranged in a side wall of the first lumen and wherein the surface is a side wall of the fluid chamber.

19. The assembly of claim 17, wherein the egress port is arranged between the first outlet and the distal end along the longitudinal axis.

20. The assembly of claim 17, wherein the fluid chamber is inflatable.
